(19) United States
(12) Patent Application Publication
(10) Pub. No.: US 2014/0053758 A1
(43) Pub. Date: Feb. 27, 2014

(54) RESTORATIVE MATERIALS

(71) Applicants: John Nicholson, Greenwich (GB); Samantha Booth, Greenwich (GB)

(72) Inventors: John Nicholson, Greenwich (GB); Samantha Booth, Greenwich (GB)

(73) Assignee: University of Greenwich, Greenwich (GB)

(21) Appl. No.: 13/981,421

(22) PCT Filed: Jan. 24, 2012
(86) PCT No.: PCT/GB12/50147

§ 371 Date: Nov. 8, 2013

(30) Foreign Application Priority Data
Jan. 24, 2011 (GB) 1101170.7

(51) Int. Cl. A61K 6/083; A61L 24/12

(52) U.S. Cl. CPC A61K 6/0835; A61L 24/12
USPC 106/810; 106/814; 523/116

(57) ABSTRACT

The present invention relates to restorative materials, in particular to glass-ionomer cements. More particularly, the present invention relates to glass-ionomer cements having particular utility in the repair of human hard tissue, in particular as dental restorative materials and in orthopaedic surgery. We describe a composition comprising a mixture of a glass ionomer cement and zinc phosphate. Preferably, the composition comprise from 40 to 95% by weight of glass ionomer cement and from to 60% by weight of zinc phosphate. The present invention also provides a powdered composition comprising a fluorosilicate glass and deactivated zinc oxide. Such that the zinc phosphate is formed in sith by reaction between the zinc oxide and phosphoric acid.

The present invention relates to restorative materials, in particular to glass-ionomer cements. More particularly, the present invention relates to glass-ionomer cements having particular utility in the repair of human hard tissue, in particular as dental restorative materials and in orthopaedic surgery.

The glass-ionomer cement (GIC) was invented by Wilson and Kent in 1969 (GB 1316129) and is now a well established material with an important role in clinical dentistry and other fields, such as a bone replacement material. It is formed by the combination of a precursor glass in the form of an ion-leachable glass powder and an aqueous solution of a polyalkenoic acid (polyacid). The glass polyalkenoate cement has a combination of clinically attractive characteristics. It can adhere to tooth dentine and enamel as well as to base metals. The cement releases fluoride over a long period of time and this can help to prevent the formation of caries. A particular attraction is that the appearance of the material is similar to that of tooth colour and so can be matched closely with the patient's natural tooth colour. The glass component of a GIC acts as a source of ions for the cement-forming reaction, controls the translucency, setting rate and strength of the cement.

Traditionally, GICs were composed of calcium aluminosilicates but modern GICs replaces the calcium by either strontium or a combination of strontium and lanthanum, which also makes the material radio-opaque. There are a substantial number of potential glasses that could be used to produce GICs, all containing silica, alumina and an alkaline earth or rare earth oxide or fluoride. The two principal glass types are SiO₂—Al₂O₃—CaO and SiO₂—Al₂O₃—CaF₂. Many other glasses can be derived from both of these materials.

Calcium fluoride is an essential constituent in fluoride glasses but often cryolite, Na₃AlF₆, is added to lower the fusion temperature. Apart from lowering the fusion temperature, fluoride improves the handling of the cement paste; increases cement translucency and strength and has a therapeutic quality when used as a dental filling.

In fluoride glasses, the alumina to silica ratio controls the setting time of the cement. Fluoride tends to slow the setting whereas aluminium orthophosphate improves the mixing of the cement. The formation of a GIC requires the complete decomposition of the glass structure so that all of the glass ions are available for release.

It has been observed that as the SiO₂:Al₂O₃ ratio decreases, the compressive strength of the material increases. Setting time also decreases as the SiO₂:Al₂O₃ ratio decreases until phase separation occurs in the glass, which deactivates the glass. As phase separation occurs, the main phase is depleted in calcium and fluoride, which reduces its reactivity. Further acid attack will occur selectively in the remaining phase separated droplets of concentrated calcium and fluoride. It has been reported that phase separated glasses have higher compressive and flexural strengths than clear glasses.

The role of fluoride within GICs is a matter of debate. According to some, in addition to [SiO₄] and [AlO₄], such glasses contain [SiO₃F] or [AlO₃F] tetrahedra. The replacement of O²⁻ by F reduces the screening of the central cation and so strengthens the remaining cation-oxygen bonds. However, fluoride is non-bridging and thus structure-breaking. Another view on the role of fluoride suggests that metal fluorides occupy holes in the major glass network.

The polyalkenoic acid is not always present in liquid form. The acid is often supplied in dry form and blended with the glass powder so it can be activated immediately prior to use with water or an aqueous solution of tartaric acid. An increase in concentration of the polyacid increases solution viscosity. This can also lead to higher strengths but at the sacrifice of working time. The molecular weight of the polyacid affects the properties of a GIC. Strength, fracture toughness, and resistance to erosion and wear are all improved as the molecular weight of the polyacid is increased. However, the working time is decreased due to accelerated setting, limiting the maximum practical molecular weight of the polyacid to 75,000.

Reaction-controlling additives are incorporated into the GIC system to give viable setting and working times. Tartaric acid is often added to sharpen the set and increase the hardening rate. It has been shown that strength can also be improved by incorporating additives. Other multifunctional carboxylic additives have been trialled, but none have been shown to be as successful as tartaric acid.

The setting of a glass-ionomer cement occurs in several overlapping stages.
1. On mixing the glass powder and liquid acid components, the calcium aluminosilicate glass is attacked by hydrogen ions from the polyalkenoic acid and decomposes with liberation of metal ions (aluminium and calcium), fluoride (if present), and silicic acid which later condenses to form silica gel.
2. As the pH of the cement paste rises, the polyalkenoic acid ionises and is likely to create an electrostatic field which aids the migration of liberated cations into the aqueous phase.
3. As the polyalkenoic acid ionises, polymer chains uncoil as the negative charge increases and the viscosity of the paste increases. The concentration of cations increases until they condense on the polyacid chain. Desolvation occurs and insoluble salts precipitate first as a sol which then coverts to a gel. This stage represents the initial set.
4. After the initial set, the cement continues to harden as cations are increasingly bound to the polyanion chain and hydration reactions continue. This process continues and does not appear to stop however the cement is hardened and ready for testing purposes after 48 hours.

Cement formation with oxide glasses is extremely rapid and the set occurs virtually on contact between the two components making it clinically useless. If tartaric acid is added to the system, a useable cement can be formed. However, other cements are preferred because of their easier manipulation properties. Clinically the most common cements used contain fluoride and (+)-tartaric acid.

The structure of a set glass-ionomer cement can be described as ‘particles of partially degraded glass embedded in a matrix of calcium and aluminium polyalkenoates and sheathed in a layer of siliceous gel’.

The GIC behaves like a thermoplastic material when initially setting, which makes it very pliable and easy to manipulate—ideal for clinicians. Setting times at 37° C. for thickly mixed cements (for filling purposes) range on average from 2.7 to 4.7 minutes. For more thinly mixed luting agents the setting time can range from 4.5 to 6.3 minutes. Strength develops quickly and after 24 hours the cements can reach very high compressive strength values. Fracture toughness and flexural strength are clinically more significant than compressive strength. The flexural strength of a GIC can reach much higher than that of the original dental silicate cement. In general, GICs exhibit low values for flexural strength and fracture toughness when compared with the values for composite resins or dental amalgams. This makes the GIC less suitable than these materials in load-bearing or high stress situations.

The bond strength of GIC to enamel is far greater than that of GIC to dentine. Bond strength develops quickly and is complete within 15 minutes according to some studies. This property of a GIC is unique as it not only penetrates the pellicle but bonds to the debris, calciferous tooth and smear layer present after drilling.

Although the GIC is the most durable of all dental cements it is still susceptible to attack by aqueous fluids under certain conditions i.e. acid erosion, ion release and water absorption. When fully hardened, the GIC is resistant to erosion provided the solution has a pH of above 4. However, in the initial setting stage, the cement is fully susceptible to acid attack as the cations are in a soluble form, which is why a number of clinicians put varnishes on the surface while the material is maturing. When immature GICs are exposed to neutral solutions such as saliva, they release ions and absorb water. The matrix forming Al³⁺ (but not Ca²⁺) can be lost, resulting in permanent damage. Other ions often lost are sodium, fluoride and silicic acid. After storage in water the cements rapidly absorb water. As the cement ages, the absorption of water and loss of aluminium ions ceases. Fluoride and silicic acid continue to be eluted. From this the GIC can be viewed as a device for the sustained release of fluoride. The release of fluoride is biologically important as it is taken up by the adjacent tooth material possibly by the exchange of F for Off in hydroxyapatite. This uptake has the effect of improving the resistance of the tooth material to acid attack. One study showed that fluoride adsorption reduced surface energy making it more difficult for caries-promoting plaque to adhere to the surface. Fluoride release also increases mineralisation of the tooth and decreases the growth of plaque bacteria.

GICs are known for their biocompatibility their ability of the material to perform with appropriate host response in a specific application. For dental applications, glass-ionomers are in contact with hard tissue and close to the pulp. Their low setting exotherm and absence of organic eluants makes them bio compatible in this application. Their ability to release fluoride and an excellent seal are other benefits of these materials. The condition of the seal between the restoration and the tooth is extremely important. It has been shown that if harmful bacteria seep beneath the restoration secondary caries can develop. This occurrence is very high in number with amalgam fillings. The pulpal inflammation caused by a restorative has been shown to be caused by the build up of bacteria and not the chemical toxicity of the restorative. The GIC is well tolerated by living cells, although an important distinction must be made between a freshly mixed cement paste and a set restorative. Fresh cement exhibits an antimicrobial effect but it has been shown that this capacity diminishes with time. It also exhibits some cytotoxicity when freshly mixed but none when set. Both the cytotoxicity and antimicrobial properties are associated with the leachate from the cement. It has been suggested that the cause of this is the low pH and high quantity of fluoride released within the fresh material whereas others suggest the effects are due to the release of metal ions or free polyacrylic acid.

There is almost continuous development of restorative materials to seek to improve their properties. Attempts have therefore frequently been made to try to improve cement strength of GICs, including by carbon fibre or metal reinforcement, in particular by addition of silver-tin alloy to the cement matrix. However, the materials are not aesthetically pleasing. In the 1990s a resin-modified glass-ionomer cement (RMGIC) was developed. Originally developed as a base and liner, it consists of a liquid polyacid, typically poly(acrylic acid), and a photopolymerizable monomer, typically 2-hydroxyethylmethacrylate, HEMA, plus a photoinitiator which react to harden the material when a visible light beam is applied. Once the resin is cured the glass-ionomer maturation reaction continues protected by the cured resin enclosure from moisture and drying out. The addition of the resin component decreases the initial setting time as the light curing process only takes ˜40 seconds. The resin also reduces handling difficulties and substantially increases the wear resistance and physical strength of the cement which makes it a very appealing material to use in the dental industry. This enthusiastic approach to resin modified glass-ionomers has continued up to the present day with many clinical trials and research supporting this type of system.

The brittleness of glass-ionomers has been a significant drawback. The brittle nature of the material means that the distribution of air voids, microcracks and other defects within the cement lowers the strength significantly. The extent of brittleness can also be enhanced by the dehydration the cement undergoes in the oral cavity. It has been shown that low flexural strength limits the clinical use of the GIC as a permanent filling material in the posterior region. It is suggested that the strength of the material is sufficient to withstand moderate occlusal load; provided it is surrounded by tooth structure. The GIC has been deemed ideal for the modern minimal intervention type of conservative operative dentistry because it will have adequate support from the surrounding tooth structure and its inherent brittleness will be of no consequence.

Efforts for improvement have been made in several aspects, involving formation of different kinds of self-cured glass-ionomers, such as acrylic acid-itaconic acid (AA-IA) copolymers and acrylic acid-maleic acid (AA-MA) copolymers, water hardening compositions and dual setting RMGICs.

Even though there are clear improvements in the field, glass-ionomers are still in need of improved surface hardness to enable reliable use in load bearing situations. The present invention seeks to overcome this problem.

The present invention seeks to provide a modified glass-ionomer material which retains the desirable characteristics of conventional glass-ionomer cements (fluoride release, strength, adhesion to human hard tissue, biocompatibility) while improving toughness. Tougher, less brittle materials, are required in order to increase durability within dentistry, and in order to enhance load-bearing ability, notably in the restoration of posterior teeth (molars and premolars), and also in orthopaedics in the repair of bone damaged by trauma or disease.

The present invention also seeks to provide a dental restorative material with enhanced fluoride release, to aid protection of repaired teeth against further damage by dental caries; and to provide a material capable of developing ion-exchange bonding with repaired teeth, with both enamel and dentine.

The present invention also seeks to provide a material for dentistry having inherent anti-microbial properties, in order to reduce the incidence of dental caries adjacent to the repair in the tooth.

The present invention also seeks to provide a material of enhanced biocompatibility, especially for use in bone repair where this property will enhance bone re-growth and the development of a durable and functional interface between the cement and the bone.

In its broadest sense, the present invention provides a glass-ionomer cement composition comprising zinc and phosphate ions.

Zinc phosphate has been used as a dental material since 1879. The material typically comprises zinc oxide powder in which small quantities of magnesium oxide are incorporated. The powder is then reacted with phosphoric acid. The main problem with this type of cement is the setting reaction and the inability to control it. If the reaction is over vigorous, the product becomes a crystalline mass rather than a cement. To moderate the reaction between zinc oxide and phosphoric acid, the zinc oxide can be sintered at between 1000-1350° C., which deactivates and densifies the starting material by reducing the surface area and surface energy. It also alters the composition to make it non-stoichiometric. The addition of magnesium oxide promotes densification and preserves the whiteness of the powder.

The liquid component of zinc phosphate cement is an aqueous solution of phosphoric acid containing aluminium. When the two components, phosphoric acid and zinc oxide, are combined; the cement forms and sets very rapidly. The reaction is strongly exothermic and is greater than with any other dental cement. The excessive heat generated has to be dissipated whilst mixing or the cement will set prematurely. Strength develops very rapidly. It has been reported that approximately half the final strength will be attained within ten minutes of mixing, and 80% after one hour.

It has been shown that the aluminium in the phosphoric acid has a profound effect on the cement. If the aluminium is not added the material formed is a crystalline mass of hopeite with little mechanical strength. On the addition of aluminium an amorphous matrix was formed with a much higher mechanical strength.

Generally, after mixing, the zinc oxide powder is attacked by the acid solution, water acting as the reaction medium. Zinc ions are extracted and the pH at the powder-liquid interface rises. This causes aluminium phosphate or zinc aluminophosphate to precipitate as a gel at the particle surface. This gel coating moderates the reaction. Zinc ions diffuse through this layer and as the pH rises an amorphous gel is precipitated (probably as zinc aluminium phosphate). As the reaction proceeds, the cement matrix becomes more hydrated. The final cement is considered to contain mainly amorphous zinc phosphate with some crystalline hydrated zinc phosphate Zn₃(PO₄)₂.4H₂O.

Clinically, zinc phosphate is used as a luting material for the cementation of crowns and bridges. The cement suffers from the lack of the adhesive property but its reliability and speed of set has ensured its place in the dental clinicians' cabinet. Fully hardened cements have brittle characteristics. However, the materials have fairly high compressive strengths.

Accordingly, the present invention provides a composition comprising a mixture of a glass ionomer cement and zinc phosphate.

More specifically, the present invention provides a restorative composition comprising a glass ionomer cement and zinc phosphate.

Preferably, the composition comprises from 40 to 95% by weight of glass ionomer cement and from 5 to 60% by weight of zinc phosphate; more preferably, from 60 to 80% by weight of glass ionomer cement and from 20 to 40% by weight of zinc phosphate, even more preferably, from 70 to 80% by weight of glass ionomer cement and from 20 to 30% by weight of zinc phosphate.

Advantageously, the composition comprises about 75% by weight of glass ionomer cement and about 25% by weight of zinc phosphate.

The present invention also provides a composition obtainable by reacting together a glass ionomer cement precursor, a polyalkenoic acid, zinc oxide and phosphoric acid.

The present invention also provides a method of preparing a restorative composition as described above; the method comprising: i) providing a glass ionomer cement precursor glass; ii) providing a deactivated zinc oxide; iii) providing a polyalkenoic acid; and iv) providing a phosphoric acid solution.

Advantageously, the glass ionomer cement precursor glass, zinc oxide and polyalkenoic acid are provided as powdered solids; and the composition is prepared by pre-mixing the powdered solids and then mixing with the phosphoric acid solution.

Alternatively, the glass ionomer cement precursor glass and the zinc oxide are provided as a powdered mixture; the polyalkenoic acid is provided as a solution; and the powdered mixture, polyalkenoic acid solution and phosphoric acid solutions are added to the powdered mixture substantially simultaneously with mixing.

The present invention also provides a kit of parts comprising: a glass ionomer cement precursor glass; ii) deactivated zinc oxide; iii) a polyalkenoic acid; and iv) phosphoric acid solution.

Conveniently, the glass ionomer cement precursor glass and deactivated zinc oxide are provided as a powdered mixture.

The present invention also provides a powdered composition comprising a fluorosilicate glass and deactivated zinc oxide.

Preferably, the composition comprises 40-95% by weight of fluorosilicate glass and 5-60% by weight of zinc oxide.

Advantageously, the glass is a fluoroaluminosilicate glass, preferably a SiO₂—Al₂O₃—CaF₂ glass, optionally including one or more of AlPO₄, Na₃AlF₆ and metal oxide or metal fluoride radio-opacifiers.

Preferably, the powdered composition further comprising a polyalkenoic acid, more preferably, in an amount, based on the glass and zinc oxide, of 10-40% by weight.

Suitably, the polyalkenoic acid is a polymer of an ethylenically unsaturated monomer, preferably polyacrylic acid, more preferably in a molar mass range of 5,000-250,000; or a homopolymer of maleic acid, itaconic acid and/or vinyl phosphonic acid or a copolymer thereof with polyacrylic acid; or mixtures of homopolymers thereof.

Preferably, the composition further comprises phosphoric acid, more preferably, in an amount of 5-40% by weight based on the weight of glass and zinc oxide.

Advantageously, the composition further comprises tartaric acid.

Advantageously, the composition further comprises a strengthening additive, preferably a finely divided metal alloy or particulate ceramic.

Suitably, the composition further comprises an additional fluoride-containing compound to enhance fluoride release; preferably, SnF₂, NaF and/or sodium mono fluorophosphate.

A composition advantageously further comprises a finely divided bioglass filler.

The compositions are suitable for use, inter alia, as a dental restorative material; as a bone defect repairing material; and as a scaffold material in tissue engineering.

The above and other aspects of the invention will now be described in further detail, by way of example only, with reference to the following examples. The following study was carried out to try to incorporate the adhesive property of a glass-ionomer cement into zinc phosphate with the aim of achieving a material with higher compressive strengths and surface hardness values without compromising the adhesive and biocompatibility of a modem glass-ionomer.

### Materials and Methods

For our studies two different types of cement were used; Fuji IX and zinc phosphate. Fuji IX is a typical GIC and is a strontium-based tooth-coloured glass ionomer luting material of alumino-silicate glass powder which is mixed with 40-45% m/v polyacrylic acid (eg. 0.25 g powder, 0.05 g liquid). Zinc phosphate is formed from a mixture of zinc oxide and phosphoric acid (45-65% m/v; eg. 0.225 g powder and 0.125 g liquid). The characterisation and analytical techniques used in this study were Vickers Hardness, Compressive strength, ICP-OES, Ion Selective Electrode, SEM, EDS and XRD.

It will therefore be understood that this represents, in fact, a four-component mixture of glass and polyacrylic acid and zinc oxide and phosphoric acid in a 50:50 mixture.

A preliminary study was performed on a 50/50 mix of Fuji IX and zinc phosphate.

Three samples were prepared using the same moulds and cure conditions. The surface hardness of the samples was tested after 24 hour storage in an air-tight bag and the results show in Table 1.

### Surface Hardness:

TABLE 1
Vickers hardness of hybrid cement stored in air

| | Surface Hardness |
|---|---|
| Mean | 76.4 |
| S.D | 1.9 |

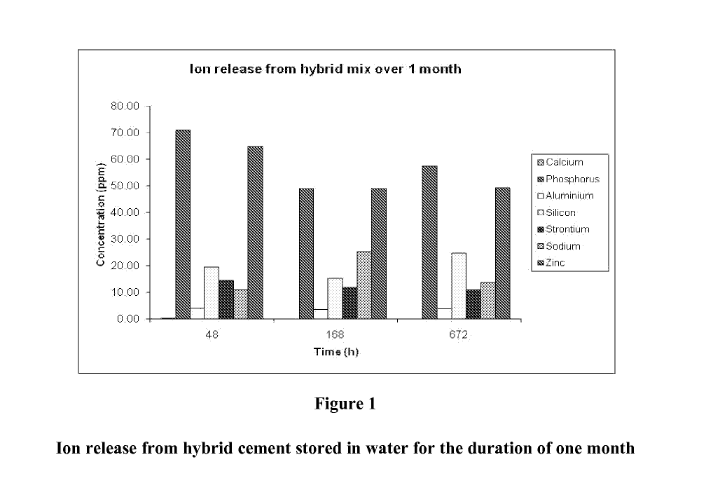

This ratio was used to produce a cement which was then subjected to 1 month of storage in water. The hardness of these specimens was tested at 48 h, 1 week and 1 month. The mean data and standard deviations are given in Table 2. Ion release analysis was also carried out on these specimens (Table 3 and illustrated in FIG. 1).

TABLE 2
Vickers hardness of the hybrid cement after storage in water

| Time in water | Mean | S.D |
|---|---|---|
| 48 h | 56.2 | 1.2 |
| 1 Week | 35.8 | 1.1 |
| 1 Month | 43.5 | 7.7 |

From the above results it can be concluded that storage in water reduces the surface hardness of the hybrid material. This may be due to an inappropriate ratio blend resulting in not enough powder reacting when mixed, producing a specimen which is fairly soluble.

TABLE 3
Ion release from hybrid cement stored in water for one month

| | | |
|---|---|---|
| 48 h | 168 h | 672 h |

| | Mean | S.D | Mean | S.D | Mean | S.D |
|---|---|---|---|---|---|---|
| Ca | 0.01 | 0.07 | b.d.l. | — | b.d.l. | — |
| P | 70.78 | 16.14 | 48.93 | 1.24 | 57.38 | 7.38 |
| Al | 3.89 | 1.88 | 3.50 | <0.01 | 3.72 | 0.64 |
| Si | 19.47 | 3.59 | 15.23 | 0.11 | 24.62 | 5.46 |
| Sr | 14.35 | 1.34 | 11.80 | 0.28 | 11.00 | 0.28 |
| Na | 10.79 | 2.14 | 25.25 | 0.14 | 13.63 | 0.79 |
| Zn | 64.66 | 20.79 | 48.98 | 3.22 | 49.10 | 1.22 |

FIG. 1 shows the ion release from hybrid cement stored in water for the duration of one month and shows that the high levels of zinc and phosphorus released could indicate that a smaller amount of zinc phosphate is appropriate for the optimum cement mix.

Cements comprising 50% Fuji IX and 50% zinc phosphate were used in the initial study and both cement components were made to the manufacturer's instructions and then combined. However, it was apparent that there was excess acid for the required amount of basic powder. An experiment was therefore carried out to determine if the acid type used (either H₃PO₄ or PAA) made a significant difference (p<0.001) to the surface hardness. 0.3 mL of either acid was incorporated into the powder during mixing and cured at 37° C. for one hour. The samples were then either left in air or distilled water for 24 h after which the surface hardness was tested. This trial was performed in triplicate. The mean data is given in Table 4 with standard deviations in parentheses.

TABLE 4
Vickers hardness of hybrid cement stored in air and water

| | Air | Water |
|---|---|---|
| H₃PO₄ | 62.83 (1.7) | 58.12 (6.8) |
| PAA | 35.55 (1.1) | 27.18 (1.7) |

From the results above it appears that by producing a cement using phosphoric acid alone higher surface hardness values are achieved. The cement was very difficult to mix without using polyacrylic acid therefore it was decided to perform another study looking at volume ratios of polyacrylic to phosphoric acid and comparing the resultant surface hardness values.

After further testing it was established that a reduced zinc phosphate content in the material resulted in a higher surface hardness as well as less zinc release. The cement tested (Table 5) had a reduced zinc phosphate content of 25%.

TABLE 5
Volume determination of H₃PO₄ and PAA and the
respective Vickers hardness results

| H₃PO₄ | PAA | Hardness |
|---|---|---|
| 0.19 | 0.15 | 56.21 |
| 0.13 | 0.15 | 44.70 |
| 0.38 | 0.15 | 67.29 |
| 0.19 | 0.20 | 35.00 |
| 0.19 | 0.10 | 31.48 |
| 0.19 | 0.05 | 88.67 |
| 0.19 | 0.00 | 60.09 |
| 0.00 | 0.30 | 72.78 |
| 0.38 | 0.05 | 54.39 |

From these results it was established that a ratio of 75% Fuji IX: 25% zinc phosphate and 0.188 mL H₃PO₄: 0.05 mL PAA was most effective at this stage in terms of surface hardness.

Whilst determining cement ratios and acid volumes, scanning electron micrograph (SEM) images were taken.

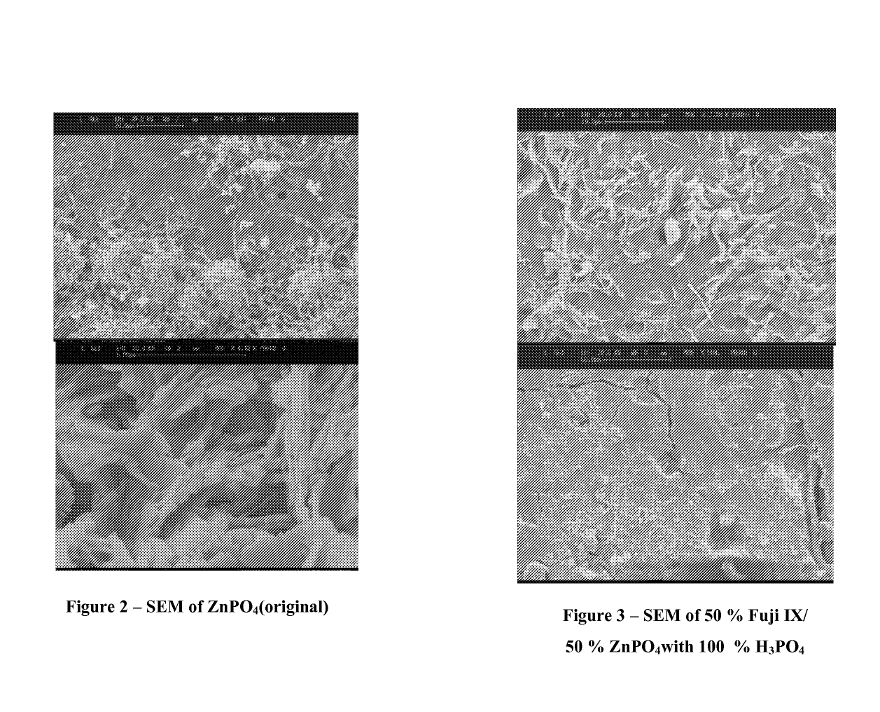

The first two trials compare the acid used. FIG. 2 is a SEM of zinc phosphate alone and shows zinc phosphate cement without any additives or alterations. The structures on the surface appear to be crystalline and resemble a structured network.

### Acid Determining Experiment:

FIG. 3 is a SEM of a 50% Fuji IX/50% zinc phosphate mixture with 100% H₃PO₄ as the binding agent. The apatite-like structures on the surface appear to have changed in morphology to shard like structures but retained their crystallinity. The crystals cover the majority of the surface but they appear to be diminished in number.

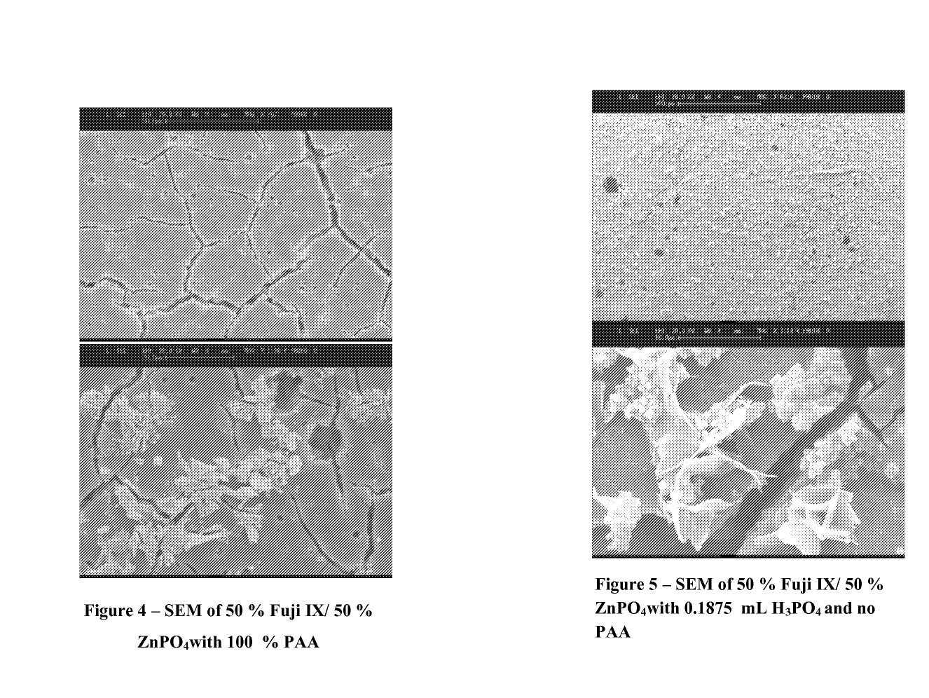

FIG. 4 is a SEM of 50% Fuji IX/50% zinc phosphate with 100% PAA (polyacrylic acid) as the binding agent. The structures on the surface appear to have changed considerably in morphology and resemble clusters. The crystals cover only a small portion of the surface and are barely visible from a lower magnification.

### Acid Volume Determination Trials:

FIG. 5 is a SEM of 50% Fuji IX/50% zinc phosphate with 0.1875 mL H₃PO₄ (no PAA) as the binding agent, but in a smaller volume than the previous study. The structures on the surface appear to have changed in morphology again and have two main types of structures—a shard like apatite and an agglomerated form. In the lower magnification image it is possible to see that the crystals cover the majority of the surface.

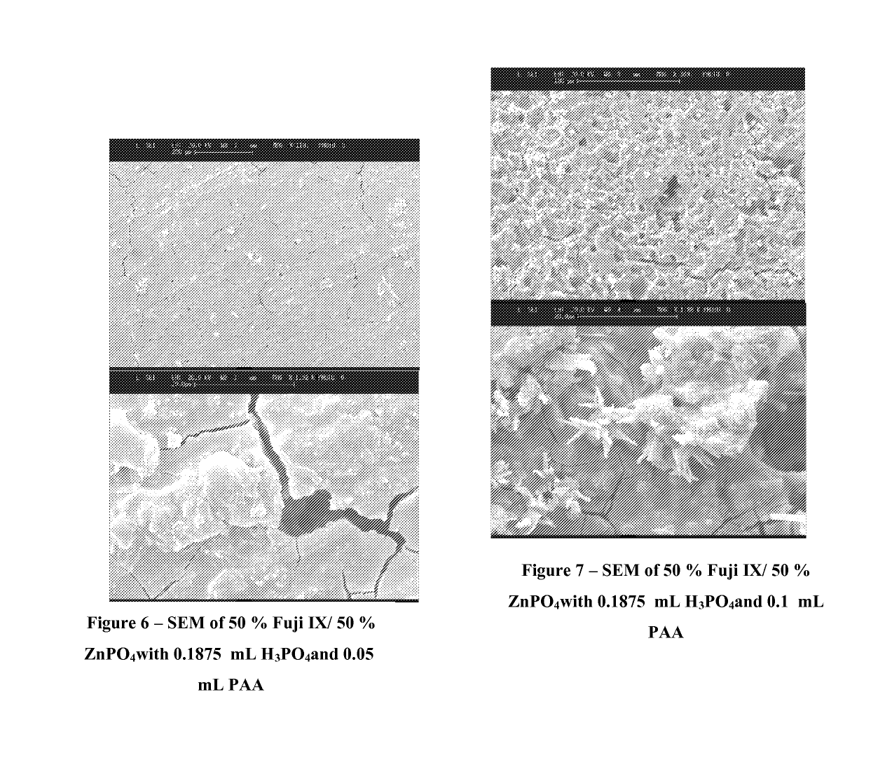

FIG. 6 is a SEM of 50% Fuji IX/50% zinc phosphate with 0.1875 mL H₃PO₄ and 0.05 mL PAA as the binding agents. The structures on the surface are vastly diminished and are only present in an agglomerated form however they are widespread over the surface.

FIG. 7 is a SEM of 50% Fuji IX/50% zinc phosphate with 0.1875 mL H₃PO₄ and 0.1 mL PAA thereby using different volumes to the previous experiment. The apatite structures on the surface have vastly increased and are present in a different shard-like form compared to the last study and are present all over the surface. At the lower magnification it appears that the acids are etching the surface of the cement which may account for the lower surface hardness.

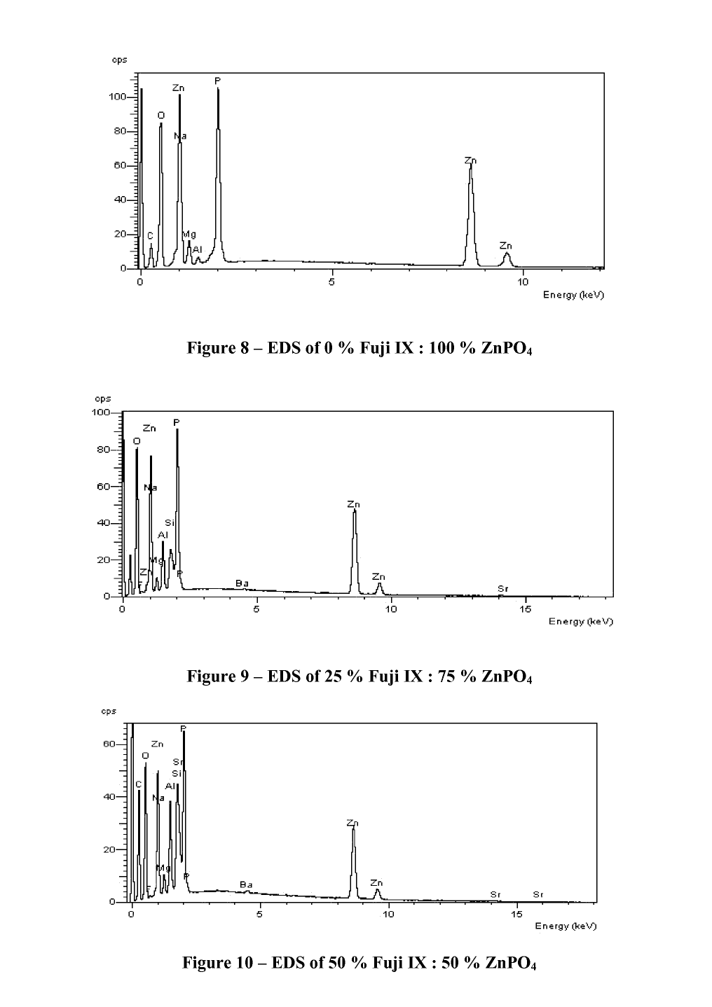

A preliminary EDS study was performed to check that an increasing amount of zinc phosphate was observed on addition to the glass-ionomer. This can be important in ensuring proper mixing when producing the cement. FIGS. 8 to 12 show spectra for a range of compositions comprising different proportions of GIC (Fuji IX) and zinc phosphate.

FIG. 8 is an EDS of 0% Fuji IX: 100% zinc phosphate.

FIG. 9 is an EDS of 25% Fuji IX: 75% zinc phosphate showing silicon, strontium and raised aluminium peaks which indicate the presence of Fuji IX.

FIG. 10 is an EDS of 50% Fuji IX: 50% zinc phosphate and compared with FIG. 9, shows elevated levels of silicon, strontium and aluminium and a reduced level of zinc, confirming a 50:50 mix.

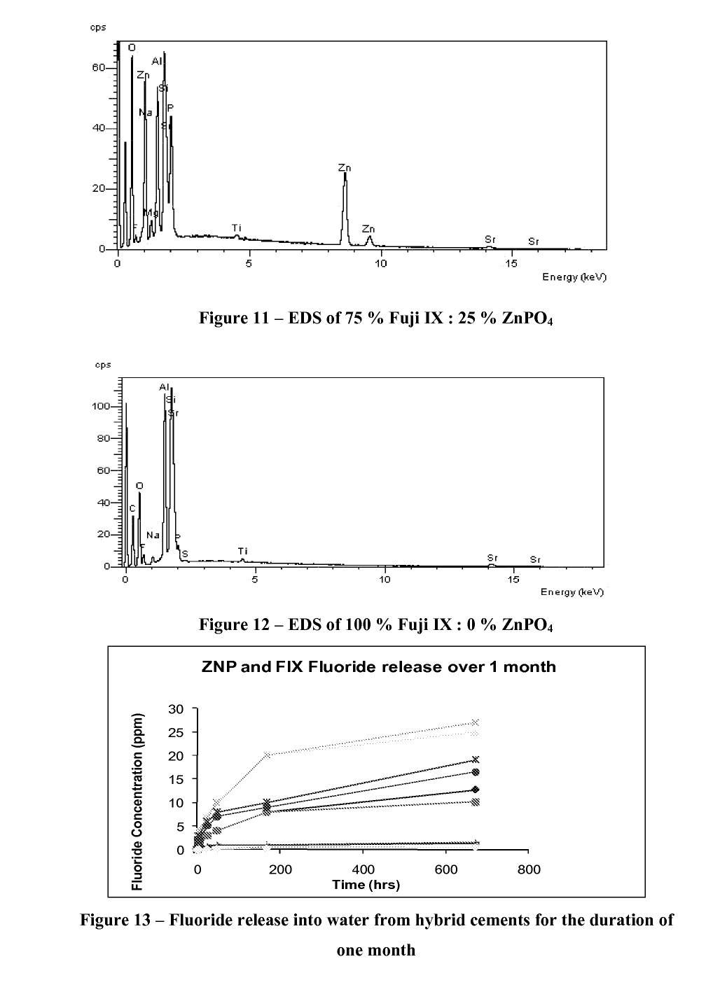

FIG. 11 is an EDS of 75% Fuji IX: 25% zinc phosphate and shows a further reduced level of zinc and phosphorus as well as a raised level of silicon, aluminium and strontium compared to the previous spectrum.

FIG. 12 is an EDS of 100% Fuji IX: 0% zinc phosphate and confirms no trace of zinc.

The spectra show that varying the ratios of glass-ionomer to zinc phosphate can be observed incrementally by EDS, indicating good mixing.

### Fluoride Release Study:

It would be important not to inhibit significantly the release of beneficial fluoride ions from the glass-ionomer component of the hybrid. Table 6 shows fluoride release results from the hybrid mixes above (results are in ppm), with two samples being prepared for each ratio mix (F=Fuji IX).

TABLE 6
Fluoride release from the hybrid cements into water for the duration of four months

| Time (h) | 0 | 1 | 2 | 3 | 4 | 5 | 24 | 48 | 168 | 672 | 2688 |
|---|---|---|---|---|---|---|---|---|---|---|---|
| 100% F1 | 0 | 0.02 | 0.60 | 0.70 | 1.00 | 1.00 | 3.00 | 4.00 | 8.00 | 12.70 | 57.50 |
| 100% F2 | 0 | 0.03 | 0.40 | 0.50 | 0.90 | 1.00 | 3.00 | 4.00 | 8.00 | 10.10 | 51.20 |
| 75% F1 | 0 | 0.50 | 2.00 | 3.00 | 4.00 | 4.00 | 7.00 | 10.00 | 20.00 | 24.90 | 102.00 |
| 75% F2 | 0 | 0.60 | 2.00 | 3.00 | 4.00 | 4.00 | 7.00 | 10.00 | 20.00 | 27.00 | 113.00 |
| 50% F1 | 0 | 0.60 | 2.00 | 3.00 | 3.00 | 3.00 | 6.00 | 8.00 | 10.00 | 19.10 | 71.20 |
| 50% F2 | 0 | 0.20 | 1.00 | 2.00 | 2.00 | 2.00 | 5.00 | 7.00 | 9.00 | 16.50 | 61.60 |
| 25% F1 | 0 | 0.01 | 0.20 | 0.30 | 0.50 | 0.50 | 0.70 | 1.00 | 1.00 | 1.52 | 5.89 |
| 25% F2 | 0 | 0.01 | 0.20 | 0.30 | 0.50 | 0.50 | 0.70 | 1.00 | 1.00 | 1.33 | 5.69 |
| 100% Z1 | 0 | 0 | 0 | 0.02 | 0.02 | 0.02 | 0.10 | 0.20 | 0.44 | 0.60 | 2.71 |
| 100% Z2 | 0 | 0 | 0 | 0 | 0 | 0.02 | 0.10 | 0.20 | 0.30 | 0.50 | 2.47 |

The results are also plotted graphically in FIGS. 13 (one month) and 14 (four months).

The results for this study show that after four months the 75%:25% Fuji IX:zinc phosphate has the greatest fluoride release, this is closely followed by the 50:50 mix and then the 100% Fuji IX. These results show that modifying a strontium based glass-ionomer by incorporating zinc phosphate significantly increases the amount of fluoride released compared with GIC alone.

### Preliminary Bioactivity Study

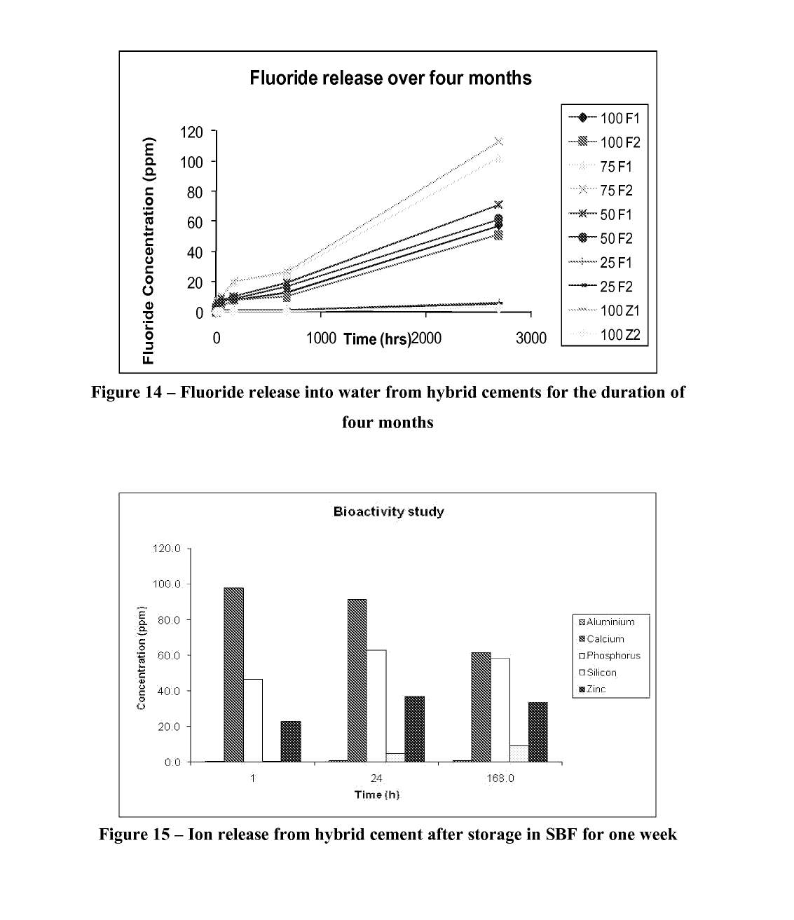

Bioactivity is a beneficial property which developing biomaterials should advantageously possess. Specimens comprising 75% Fuji IX:25% zinc phosphate were prepared, as this composition had exhibited the greatest surface hardness values and greatest fluoride release values. The samples were immersed in simulated body fluid (SBF) for 1 h, 24 h and 1 week to check for bioactivity. The results are shown graphically in FIG. 15 and tabulated in Table 7.

TABLE 5.7
Ion release from hybrid cement after storage in SBF for one week

| | | |
|---|---|---|
| 1 h | 24 h | 1 week |

| | Mean | S.D | Mean | S.D | Mean | S.D |
|---|---|---|---|---|---|---|
| Al | 0.32 | 0.05 | 0.75 | 0.11 | 0.88 | 0.19 |
| Ca | 97.81 | 0.34 | 91.43 | 0.55 | 61.62 | 0.25 |
| P | 46.42 | 0.57 | 62.99 | 0.44 | 58.31 | 0.03 |
| Si | 0.55 | 0.05 | 4.56 | 0.26 | 9.33 | 1.08 |
| Zn | 22.94 | 2.61 | 36.91 | 2.15 | 33.79 | 3.14 |

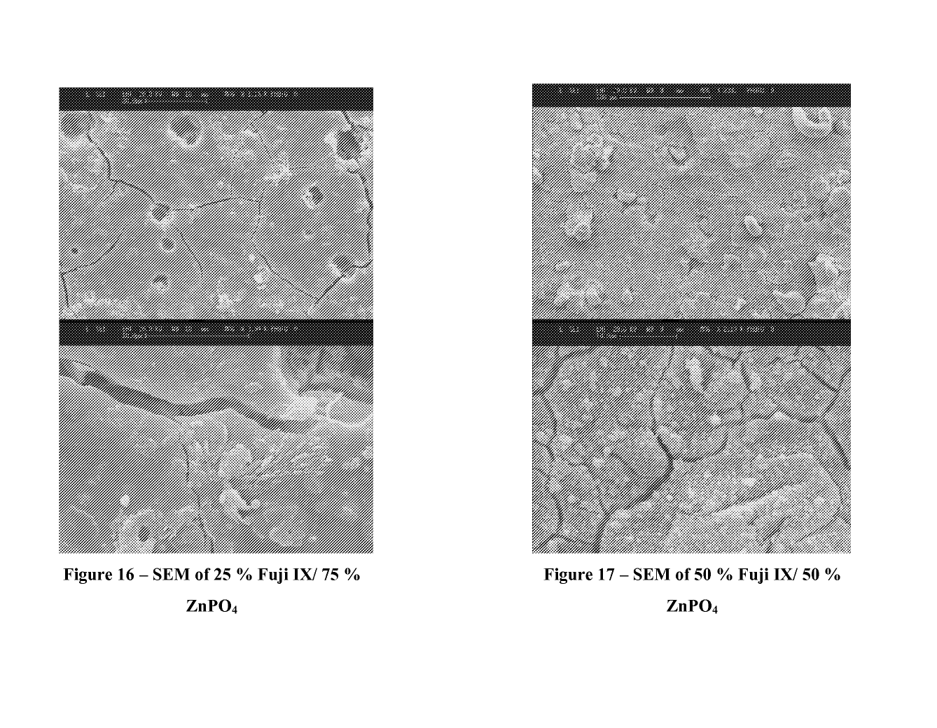

FIG. 15 shows the ion release from hybrid cement after storage in SBF for one week and indicates that, over time, the 75:25 hybrid takes up calcium ions from the SBF. This is a good indication for bioactivity. SEM studies were carried out to determine if the cements had a different morphology once they had been stored in SBF. The micrographs are shown in FIGS. 16 to 19.

The other hybrids were also immersed in SBF and SEM images were taken and similar bioactivity was confirmed.

FIG. 16 is a SEM of 25% Fuji IX/75% zinc phosphate using both phosphoric and polyacrylic acids as the binding agents. The deposits formed on the surface have no specific structures.

FIG. 17 is a SEM of 50% Fuji IX/50% zinc phosphate using both phosphoric and polyacrylic acids as the binding agents. The surface is very uneven and has deposits of different morphologies. At a higher magnification it is possible to see that the surface is coated in a deposit of what is taken to be calcium phosphate.

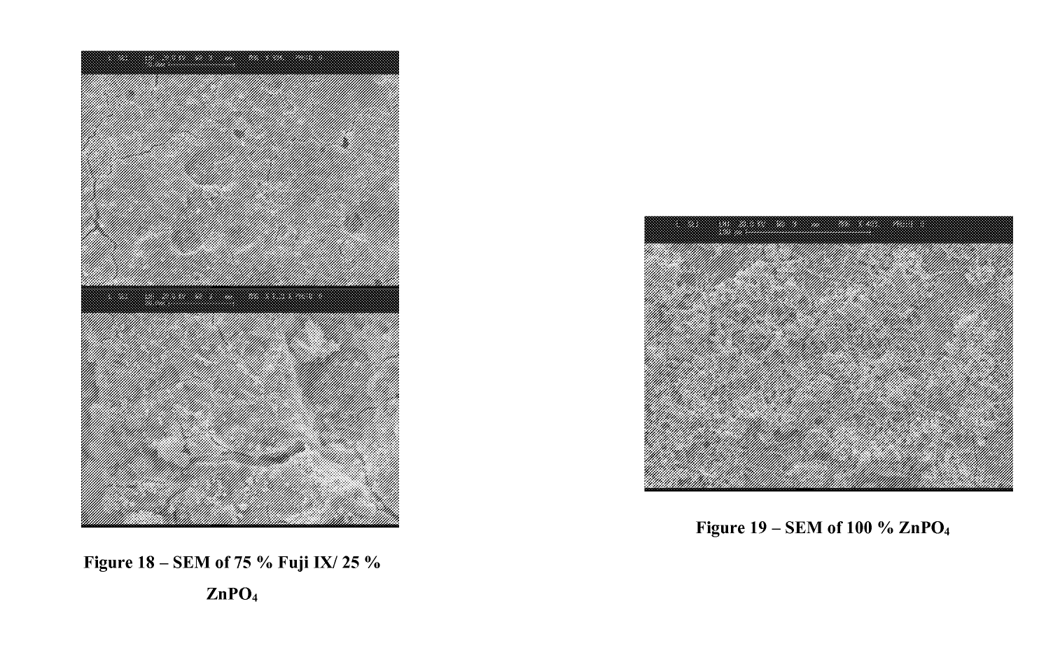

FIG. 18 is a SEM of 75% Fuji IX/25% zinc phosphate using both phosphoric and polyacrylic acids proportionally as the binding agents. The surface is uneven and has deposits of different morphologies.

FIG. 19 is a SEM of 100% zinc phosphate stored in SBF for 1 month. The surface appears to be relatively unchanged by the storage conditions and there is no apparent calcium phosphate deposition.

### Refinement

From analysis of the above data, it was decided to investigate the properties of compositions having proportions of GIC above the 75% indicated as preferable in the trials so far. It was hypothesised that the compositions may produce increased surface hardness values.

The hardness values are given in Table 8.

TABLE 8
Vickers hardness

| % Fuji IX | % Zinc Phosphate | Mean | S.D |
|---|---|---|---|
| 80 | 20 | 92.8 | 2.9 |
| 85 | 15 | 80.3 | 1.9 |
| 90 | 10 | 84.0 | 4.4 |
| 95 | 5 | 76.3 | 2.5 |

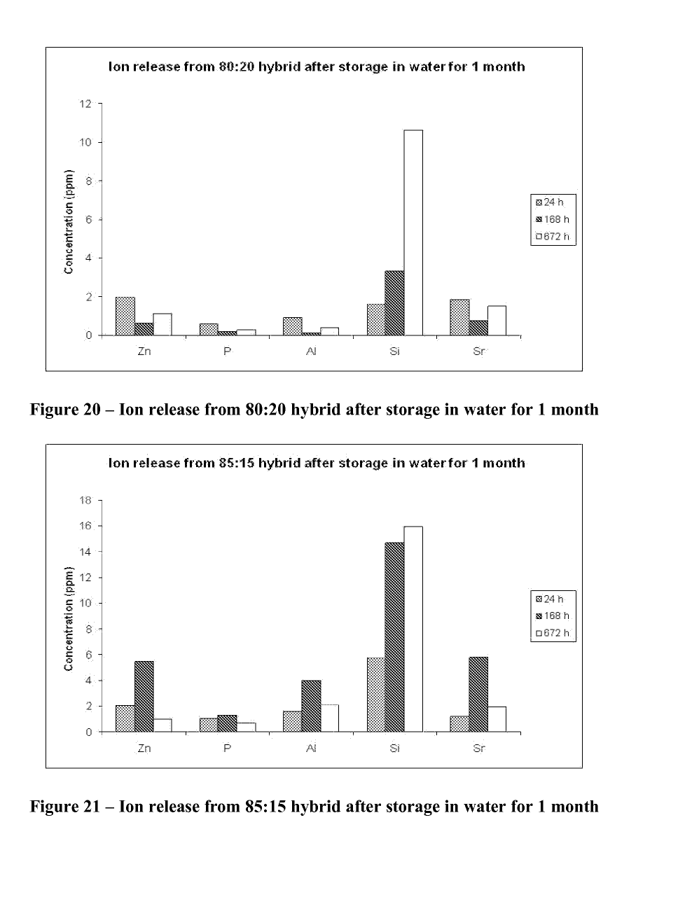

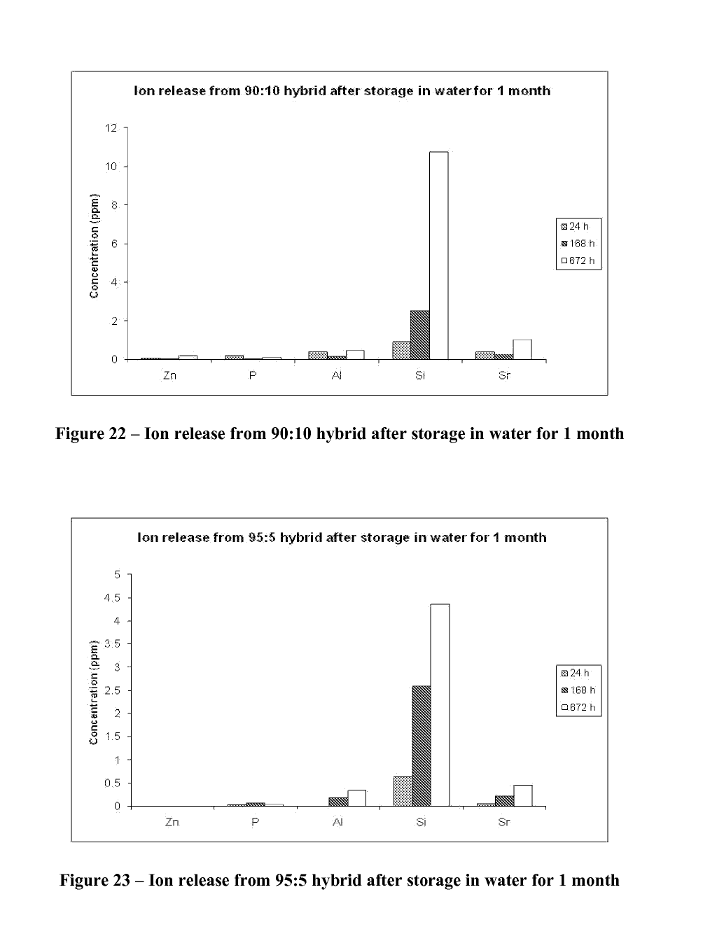

The extent of ion release from the hybrids was then assessed using triplicate sets of the hybrid cements. Samples were stored in 10 mL of distilled water at 37° C. for 24 h, 168 h or 1 month. Once the time periods had elapsed the samples were removed and the remaining water was analysed for ion release. The results are shown in Table 9 and illustrated in FIG. 20; Table 10 and FIG. 21; Table 11 and FIG. 22; and Table 12 and FIG. 23 respectively.

TABLE 9
Ion release for 80:20 hybrid

| | | | |
|---|---|---|---|
| 80:20 | 24 h | Week | Month |

| Ion | Mean | S.D | Mean | S.D | Mean | S.D |
|---|---|---|---|---|---|---|
| Zn | 1.98 | 0.06 | 0.64 | <0.01 | 1.10 | <0.01 |
| P | 0.59 | <0.01 | 0.19 | <0.01 | 0.30 | 0.02 |
| Al | 0.92 | 0.06 | 0.13 | <0.01 | 0.38 | <0.01 |
| Si | 1.62 | <0.01 | 3.34 | 0.02 | 10.62 | 0.09 |
| Sr | 1.86 | <0.01 | 0.74 | <0.01 | 1.51 | <0.01 |

Compared with the preliminary studies, the ion release for an 80% GIC: 20% zinc phosphate mixture appears to be reduced at lower zinc phosphate proportions. Even after one month the silicon release from the cement is still under 12 ppm. The early release of zinc from the cement is below 2 ppm and reduces over time.

TABLE 10
Ion release for 85:15 hybrid

| | | | |
|---|---|---|---|
| 85:15 | 24 h | Week | Month |

| Ion | Mean | S.D | Mean | S.D | Mean | S.D |
|---|---|---|---|---|---|---|
| Zn | 2.05 | 0.03 | 5.46 | 0.08 | 1.03 | <0.01 |
| P | 1.05 | <0.01 | 1.31 | 0.04 | 0.69 | <0.01 |
| Al | 1.59 | <0.01 | 3.99 | 0.07 | 2.12 | 0.05 |
| Si | 5.76 | 0.09 | 14.70 | 0.04 | 15.95 | 0.39 |
| Sr | 1.20 | <0.01 | 5.82 | <0.01 | 1.97 | <0.01 |

The silicon release from the 85:15 hybrid (FIG. 21) is higher than the 80:20 hybrid, however this value could be anticipated due to the higher amount of silicon in the cement. The higher amount of zinc being released is surprising as there should be less zinc in the material to start with. This could be an indication of interference, due to the increasing amount of Fuji IX, between the zinc oxide and phosphoric acid setting reaction.

TABLE 11
Ion release for 90:10 hybrid

| | | | |
|---|---|---|---|
| 90:10 | 24 h | Week | Month |

| Ion | Mean | S.D | Mean | S.D | Mean | S.D |
|---|---|---|---|---|---|---|
| Zn | 0.07 | <0.01 | 0.03 | <0.01 | 0.19 | <0.01 |
| P | 0.19 | <0.01 | 0.03 | <0.01 | 0.08 | <0.01 |
| Al | 0.39 | <0.01 | 0.15 | <0.01 | 0.45 | <0.01 |
| Si | 0.91 | <0.01 | 2.25 | 0.05 | 10.75 | 0.18 |
| Sr | 0.38 | 0.02 | 0.24 | <0.01 | 1.02 | <0.01 |

Similarly to the 80:20 hybrid; the 90:10 hybrid (FIG. 22) shows elevated release after 1 month of silicon, this value is less than 12 ppm and still shows good retention in the general bulk of material. There is minimal zinc release which also shows good retention.

TABLE 12
Ion release for 95:5 hybrid

| | | |
|---|---|---|
| 24 h | Week | Month |

| 95:5 | Mean | S.D | Mean | S.D | Mean | S.D |
|---|---|---|---|---|---|---|
| Zn | b.d.l. | — | b.d.l. | — | b.d.l. | — |
| P | 0.02 | <0.01 | 0.07 | <0.01 | 0.04 | 0.07 |
| Al | b.d.l. | — | 0.17 | <0.01 | 0.34 | <0.01 |
| Si | 0.63 | <0.01 | 2.59 | 0.02 | 4.36 | 0.03 |
| Sr | 0.06 | <0.01 | 0.22 | <0.01 | 0.45 | 0.02 |

All ion release for the 95:5 hybrid (FIG. 23) is reduced when compared with the other hybrids. This may be due to the material becoming structurally more like the original glass-ionomer, thus becoming more tightly bound and retaining the ions.

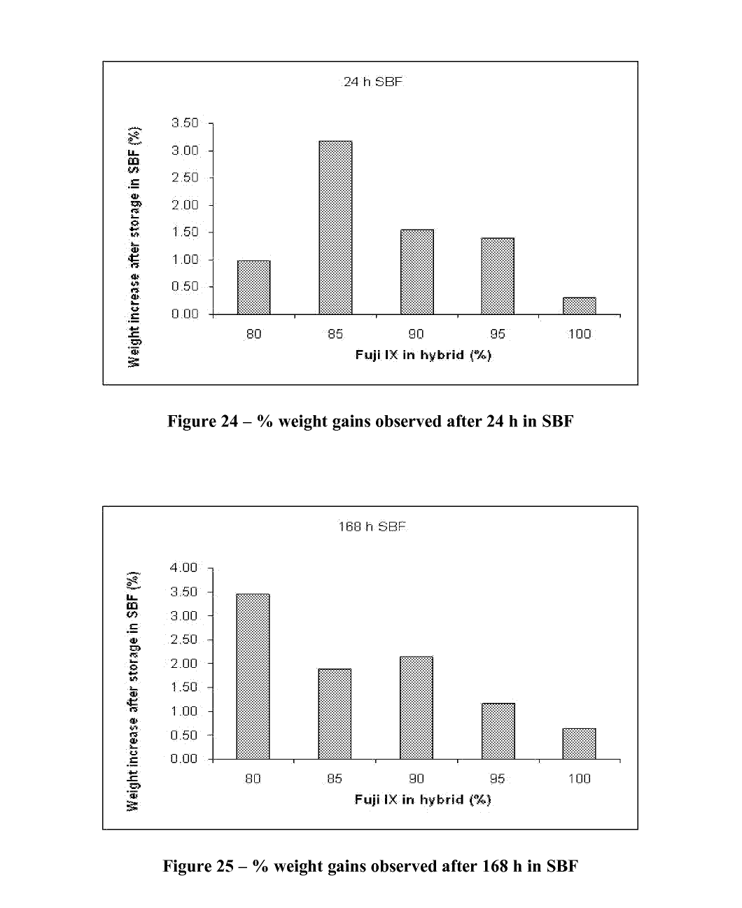

Bioactivity was then assessed. Triplicate sets of specimens were then prepared, weighed and stored in 10 mL SBF which was prepared using the Kokubo method for either 24 or 168 h. Once this time had elapsed the samples were removed, reweighed and stored for SEM analysis, the remaining SBF was diluted up to 30 mL and then analysed by ICP. The following results were obtained (Tables 13 and 14 and FIGS. 24 and 25, for the varying proportions after 24 hours and 168 hours).

TABLE 13
% weight gains observed after 24 h in SBF

| 24 h | 80:20 | 85:15 | 90:10 | 95:5 | 100 |
|---|---|---|---|---|---|
| Mean % gain | 0.97 | 3.18 | 1.54 | 1.40 | 0.30 |
| S.D | 0.10 | 0.29 | 0.13 | 0.02 | 0.04 |

TABLE 14
% weight gains observed after 168 h in SBF

| 168 h | 80:20 | 85:15 | 90:10 | 95:5 | 100 |
|---|---|---|---|---|---|
| Mean % gain | 3.45 | 1.89 | 2.15 | 1.16 | 0.65 |
| S.D | 0.42 | 0.47 | 0.18 | 0.07 | 0.04 |

It is easier to see the general trend of weight gain for the samples stored in SBF for 1 week. The samples containing a higher content of zinc phosphate appear to generally gain more weight. This is a very good indication of bioactivity however the weight gain could also be due simply to the hybrids becoming more hydrated.

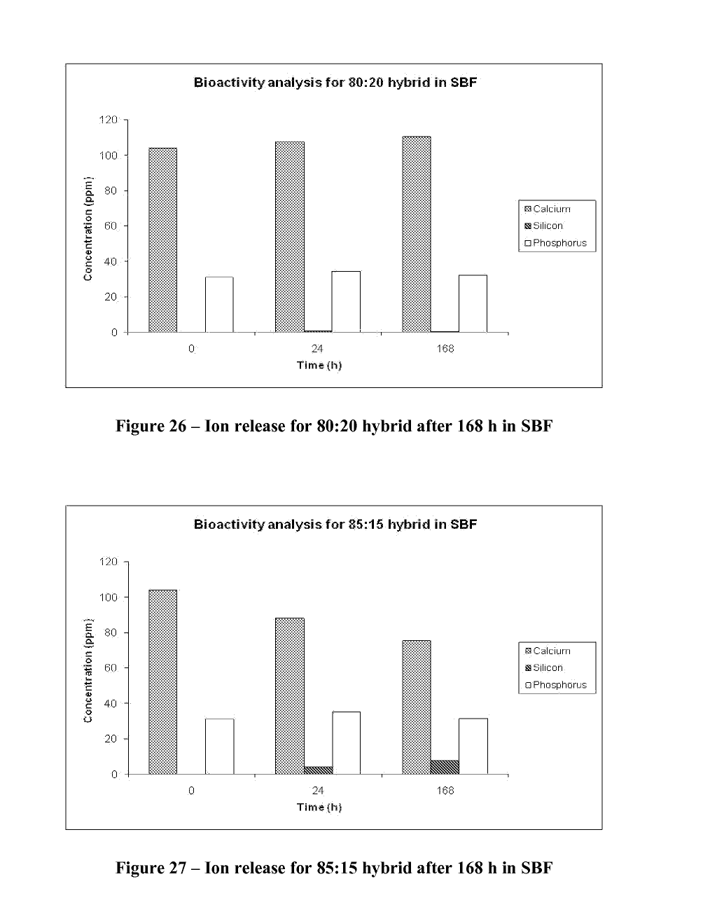

Ion-release was then assessed and the results for the various compositions given in Tables 15 to 19 and FIGS. 26 to 30.

TABLE 15
Ion release observed from 80:20 hybrid after 168 h in SBF

| Time in SBF (h) | | |
|---|---|---|
| 0 | 24 | 168 |

| | Mean | S.D | Mean | S.D | Mean | S.D |
|---|---|---|---|---|---|---|
| Ca | 104.00 | 0.26 | 107.79 | 1.59 | 110.49 | 0.31 |
| Si | b.d.l. | — | 0.73 | 0.32 | 0.20 | 0.05 |
| P | 31.00 | 0.45 | 32.2 | 1.05 | 32.37 | 1.05 |

It is unclear from the results (FIG. 26) as to whether the 80:20 cements take up any calcium or phosphorus. The weight gain observed after 168 hours is more likely to be due to hydration than to bioactivity. The cement contains a large amount of phosphorus from the phosphoric acid, making it more difficult to measure the uptake as there is more than likely to be release from the unreacted acid component.

TABLE 16
Ion release observed from 85:15 hybrid after 168 h in SBF

| Time in SBF (h) | | |
|---|---|---|
| 0 | 24 | 168 |

| | Mean | S.D | Mean | S.D | Mean | S.D |
|---|---|---|---|---|---|---|
| Ca | 104.00 | 0.26 | 88.23 | 0.87 | 75.38 | 5.71 |
| Si | b.d.l. | — | 4.19 | 0.53 | 7.79 | 7.37 |
| P | 31.00 | 0.45 | 35.18 | 0.44 | 31.36 | 2.06 |

FIG. 27 shows ion release for 85:15 hybrid after 168 h in SBF and again it is difficult to see any significant uptake of Ca or P.

TABLE 17
Ion release observed from 90:10 hybrid after 168 h in SBF

| Time in SBF (h) | | |
|---|---|---|
| 0 | 24 | 168 |

| | Mean | S.D | Mean | S.D | Mean | S.D |
|---|---|---|---|---|---|---|
| Ca | 104.00 | 0.26 | 108.98 | 3.56 | 105.10 | 0.85 |
| Si | b.d.l. | — | 0.06 | 0.07 | 0.08 | 0.01 |
| P | 31.00 | 0.45 | 37.90 | 1.76 | 38.79 | 0.17 |

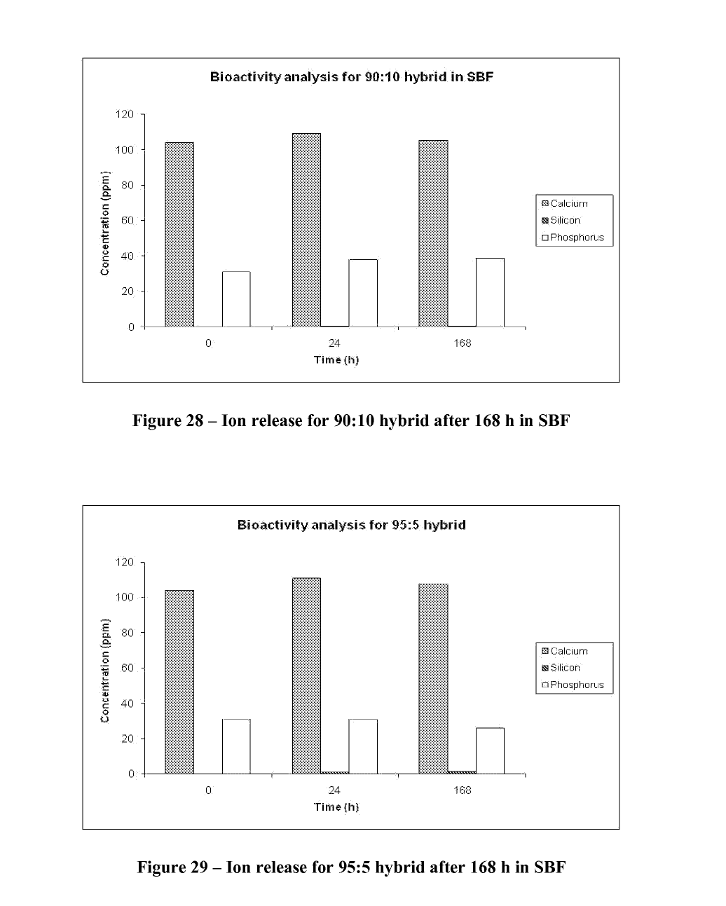

There appears (FIG. 28) to be no calcium uptake from the SBF by the 90:10 hybrid, after 168 hours in SBF, only release of phosphorus from the material.

TABLE 18
Ion release observed from 95:5 hybrid after 168 h in SBF

| Time in SBF (h) | | |
|---|---|---|
| 0 | 24 | 168 |

| | Mean | S.D | Mean | S.D | Mean | S.D |
|---|---|---|---|---|---|---|
| Ca | 104.00 | 0.26 | 110.73 | 3.34 | 107.60 | 5.95 |
| Si | b.d.l. | — | 1.13 | 0.53 | 1.31 | 0.71 |
| P | 31.00 | 0.45 | 30.98 | 0.15 | 25.95 | 0.62 |

Again there appears to be no calcium uptake by the cement in the 95:5 hybrid after 168 hours in SBF (FIG. 29). However, it is clear to see the levels of phosphorus in the previous samples have no correlation to the quantity of zinc phosphate in the material.

TABLE 19
Ion release observed from Fuji IX after 168 h in SBF

| Time in SBF (h) | | |
|---|---|---|
| 0 | 24 | 168 |

| | Mean | S.D | Mean | S.D | Mean | S.D |
|---|---|---|---|---|---|---|
| Ca | 104.00 | 0.26 | 100.65 | 0.86 | 100.82 | 1.47 |
| Si | b.d.l. | — | 0.91 | 0.42 | 0.10 | 0.01 |
| P | 31.00 | 0.45 | 34.47 | 0.72 | 34.68 | 1.38 |

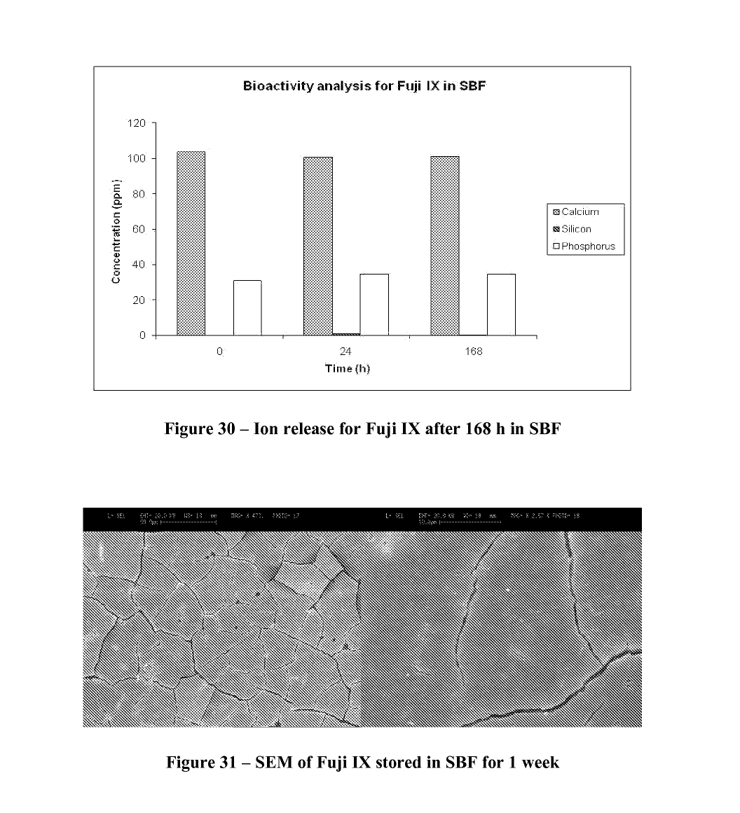

Fuji IX alone appears (FIG. 30) to have an insignificant uptake of calcium ions from the SBF after 168 hours. The bioactivity studies are fairly inconclusive as the phosphorus released from the specimens interferes with the results.

SEM results for the bioactivity study on the refined specimens are shown in FIGS. 31 to 35:

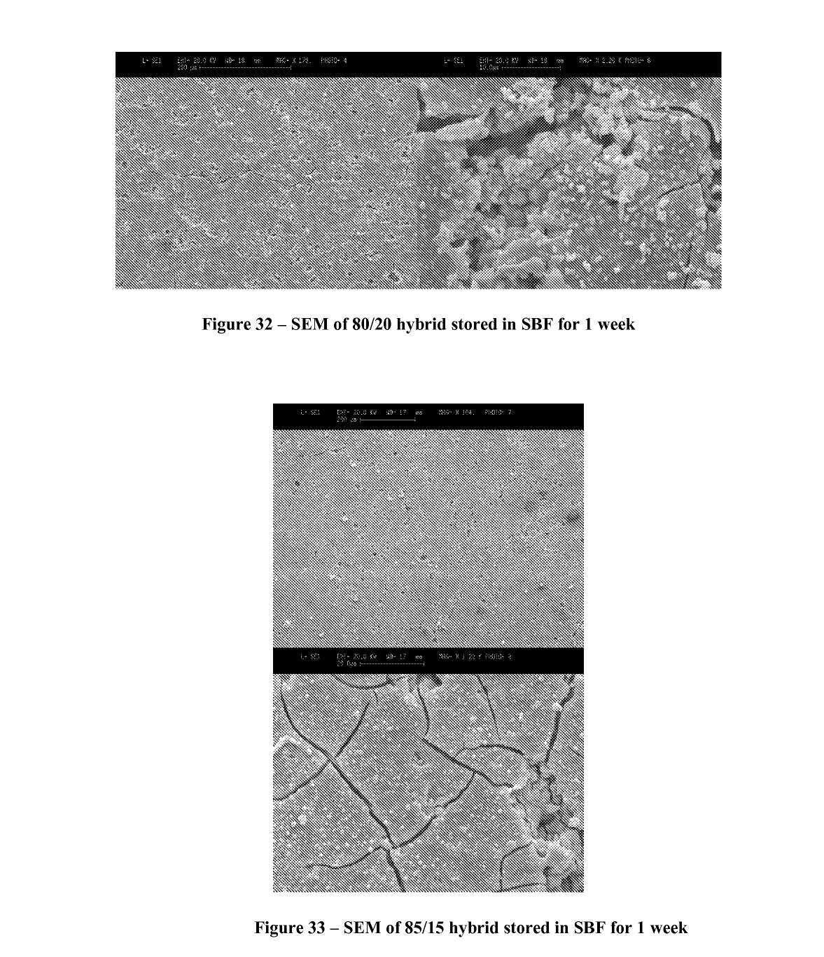

Fuji IX appears to be unaffected by the storage in SBF for 1 week (FIG. 31), the surface is smooth with just a few craters and cracks from the desiccation process. The surface of the 80:20 hybrid had been significantly altered after 1 week in SBF (FIG. 32). There are apatite structures present on the surface which nucleate around the pits in the surface of the cement indicating good biocompatibility and bioactivity.

The micrographs of FIG. 33 show that the morphology of the 85:15 cement has been modified too after 1 week of storage in SBF. When compared with the 100% Fuji IX sample stored in SBF, this specimen has craters which are similar but at a higher magnification there are deposits on the surface that are unlike the original apatite crystals, suggesting that the cement has been modified by the SBF.

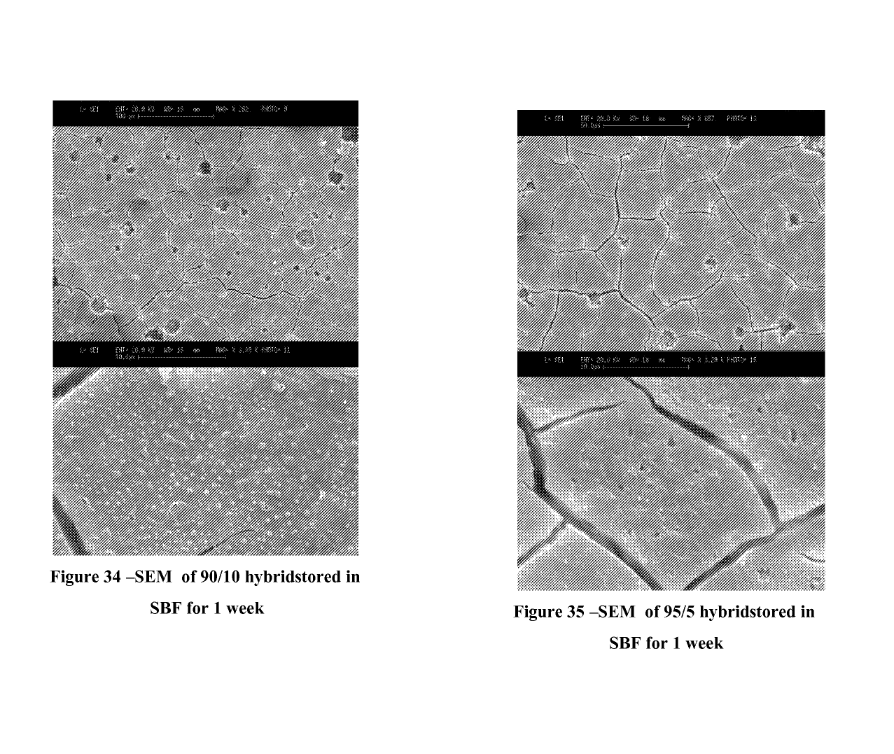

The 90:10 hybrid cement appears (FIG. 34) to look more like the original Fuji IX sample at a low magnification however at a high magnification there are still structures on the surface of unknown composition.

FIG. 35 is an SEM of 95/5 hybrid stored in SBF for 1 week and these images have the closest resemblance to the original Fuji IX sample as they contain the most glass-ionomer and the least zinc phosphate. Even with the very low amount of zinc phosphate in the sample there is still a small surface deposit present.

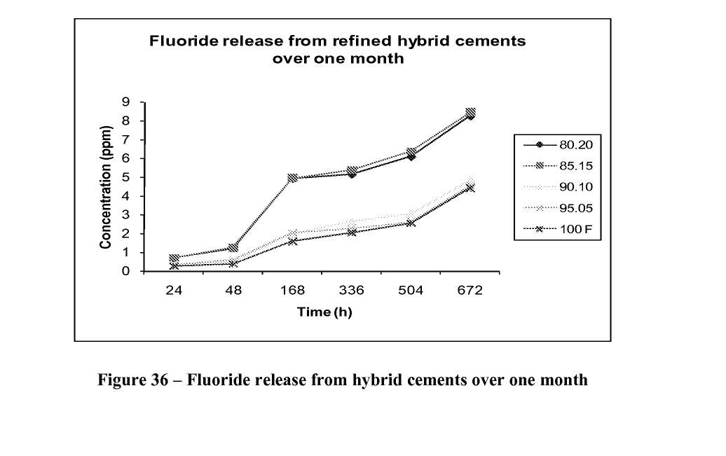

Fluoride release studies were then carried out on the further cements. The results are shown in Table 20 and FIG. 36.

TABLE 20
Fluoride release from hybrid cements over a period of 1 month

| Time in | 80:20 | | 85:15 | | 90:10 | | 95:5 | | 100 | |
|---|---|---|---|---|---|---|---|---|---|---|
| water | Mean | S.D | Mean | S.D | Mean | S.D | Mean | S.D | Mean | S.D |
| 24 | 0.68 | 0.27 | 0.69 | 0.11 | 0.29 | 0.02 | 0.31 | 0.05 | 0.25 | 0.01 |
| 48 | 1.21 | 0.44 | 1.23 | 0.23 | 0.48 | 0.06 | 0.56 | 0.10 | 0.37 | 0.06 |
| 168 | 4.90 | 0.89 | 4.93 | 0.65 | 1.97 | 0.29 | 2.03 | 0.25 | 1.57 | 0.23 |
| 336 | 5.13 | 0.31 | 5.37 | 0.65 | 2.63 | 0.32 | 2.27 | 0.21 | 2.03 | 0.06 |
| 504 | 6.07 | 0.31 | 6.37 | 1.15 | 3.03 | 0.25 | 2.60 | 0.20 | 2.53 | 0.06 |
| 672 | 8.23 | 0.25 | 8.43 | 1.07 | 4.83 | 0.06 | 4.53 | 0.32 | 4.40 | 0.44 |

It can be seen that by incorporating zinc phosphate into a glass-ionomer, elevated levels of fluoride release can be achieved. The results correspond incrementally to the increasing level of zinc phosphate in the cement. In theory as there is no fluoride in zinc phosphate cement there should be less fluoride released the more zinc phosphate that there is in the material. It is clear that the process is not as straightforward as it may first appear as this is not shown by our results.

X-Ray diffraction patterns were obtained to assess the crystallinity within each of the refined materials and it was apparent that incorporating zinc phosphate makes the resulting hybrid more crystalline. There is also evidence that the hybrid may contain zincite and hopeite, as well as the amorphous glass from the glass-ionomer.

### Antimicrobial Study

A preliminary antimicrobial study was carried out on the compositions. Zone of inhibition studies were carried out using agar plates spread with Bacteroides species. (ATCC 49057) and Actinomyces ordontolyticus (ATCC 17929). Plates contained two specimens and were left in an incubator at 37° C. for 48 h under anaerobic conditions. Zones were observed around both the 80:20 and 85:15 hybrids. There was no zone observed for the 90:10 and 95:5 hybrids. This is most probably due to the small percentage of zinc in these two hybrid materials not diffusing out from the hybrid in a sufficient quantity whereas with the higher percentage zinc-containing hybrids the zinc is more likely to have diffused to the surface of the cement. As no cell count was performed and so the concentration of bacteria was not determined. However, the same concentration was used on all samples and a difference in microbial action determined empirically.

Accordingly, it has been determined by our research that hybrid restorative materials comprising a glass-ionomer cement and zinc phosphate provides advantageous results over the use of either material alone. Fluoride release is enhanced compared with GIC alone as is surface hardness. The low ion (Zn, P, Al, Si, Sr) release in water indicates good entrapment of ions within the matrix and therefore improved maturation of the cement. The SEM results show formation of apatite structures on the surface of the cement at the preferred zinc phosphate compositional levels than is found with pure GIC, indicating enhanced bioactivity and binding clinically to tooth or bone structures. The apatite structures are also more rounded than those formed on zinc phosphate alone. The hybrid material does not lose the antimicrobial properties of zinc phosphate.

The invention comprises a mixture of glass-ionomer and zinc phosphate dental cements. Mixing of these materials leads to the formation of a set cement that has reasonable aesthetics, enhanced fluoride release and enhanced biocompatibility compared with conventional glass ionomers. Mechanically, it is tougher (less brittle) than conventional glass-ionomer cements, and it also has the ability to bond to human hard tissue, especially dentine and enamel.

In the present application, the terms ‘in use’, ‘in situ’ and ‘at the time of use’ are intended to refer to the point in time at which the composition is mixed and then used by the practioner, such as the dentist.

1. A composition comprising a mixture of a glass ionomer cement and zinc phosphate, wherein the zinc phosphate is prepared, in use, by reaction of zinc oxide and phosphoric acid; and wherein the glass ionomer cement is formed in use.

2. A composition as claimed in claim 1 comprising from 40 to 95% by weight of glass ionomer cement and from 5 to 60% by weight of zinc phosphate.

3. A composition as claimed in claim 1 comprising from 60 to 80% by weight of glass ionomer cement and from 20 to 40% by weight of zinc phosphate.

4. A composition as claimed in claim 3 comprising from 70 to 80% by weight of glass ionomer cement and from 20 to 30% by weight of zinc phosphate.

5. A composition as claimed in claim 4 comprising about 75% by weight of glass ionomer cement and about 25% by weight of zinc phosphate.

6. A composition as claimed in claim 1 claim wherein the glass ionomer cement is formed in situ by reaction of a precursor glass and a polyalkenoic acid.

7. A composition obtainable by reacting together a glass ionomer cement precursor, a polyalkeonoic acid, zinc oxide and phosphoric acid.

8. A precursor composition for the composition for claim 1, the precursor composition comprising a mixture of fluorosilicate glass and deactivated zinc oxide.

9. A composition as claimed in claim 8 comprising 40-95% by weight of fluorosilicate glass and 5-60% by weight of zinc oxide.

10. A composition as claimed in claim 6 wherein the glass is a fluoroaluminosilicate glass, preferably a SiO₂—Al₂O₃—CaF₂ glass, optionally including one or more of AlPO₄, Na₃AlF₆ and metal oxide or metal fluoride radio-opacifiers.

11. A composition as claimed in claim 8 further comprising a polyalkenoic acid.

12. A composition as claimed in claim 11 comprising polyalkenoic acid in an amount, based on the glass and zinc oxide, of 10-40% by weight.

13. A composition as claimed in claim 6, wherein the polyalkenoic acid is a polymer of an ethylenecally unsaturated monomer, preferably polyacrylic acid, more preferably in a molar mass range of 5,000-250,000; or a homopolymer of maleic acid, itaconic acid and/or vinyl phosphonic acid or a copolymer thereof with polyacrylic acid; or mixtures of homopolymers thereof.

14. A composition as claimed in claim 7, wherein the polyalkenoic acid is in solid form in admixture with the fluorosilicate glass and the zinc oxide.

15. A composition as claimed in claim 7 wherein the polyalkenoic acid is a solution of polyalkenoic acid.

16. A composition as claimed in claim 8 further comprising phosphoric acid.

17. A composition as claimed in claim 16 comprising phosphoric acid in an amount of 5-40% by weight based on the weight of glass and zinc oxide.

18. A composition as claimed in claim 1 further comprising tartaric acid.

19. A composition as claimed in claim 1 comprising a strengthening additive, preferably a finely divided metal alloy or particulate ceramic.

20. A composition as claimed in claim 1 further comprising an additional fluoride-containing compound to enhance fluoride release.

21. A composition as claimed in claim 20 wherein the additional fluoride-containing compound is SnF₂, NaF and/or sodium monofluorophosphate.

22. A composition as claimed in claim 1 further comprising a finely divided bioglass filler.

23-25. (canceled)

26. A method of preparing a restorative composition as claimed in claim 1; the method comprising:
i) providing a glass ionomer cement precursor glass;
ii) providing a deactivated zinc oxide;
iii) providing a polyalkenoic acid; and
iv) providing a phosphoric acid solution.

27-28. (canceled)

29. A kit of parts comprising:
(i) a glass ionomer cement precursor glass;
(ii) deactivated zinc oxide;
(iii) a polyalkenoic acid; and
(iv) phosphoric acid solution.

30-31. (canceled)
